(19) United States
(12) Patent Application Publication
(10) Pub. No.: US 2005/0228261 A1
(43) Pub. Date: Oct. 13, 2005

(54) Magnetic resonance imaging method

(75) Inventors: Michael Huber, Teufen (CH); Sebastian Kozerke, Hedingen (CH); Peter Boesiger, Ennetbaden (CH)

Correspondence Address: PHILIPS INTELLECTUAL PROPERTY & STANDARDS, 595 MINER ROAD, CLEVELAND, OH, 44143, US

(73) Assignee: Koninklijke Philips Electronics N.V., Eindhoven (NL)

(21) Appl. No.: 10/514,511

(22) PCT Filed: May 12, 2003
(86) PCT No.: PCT/IB03/01987

§ 371 Date: Nov. 12, 2004

(30) Foreign Application Priority Data
May 13, 2002 (EP) 02076839.6
May 13, 2002 (EP) 02076840.4

(51) Int. Cl. A61B 5/05

(52) U.S. Cl. 600/410; 600/407; 600/419; 600/422; 600/423

(57) ABSTRACT

A novel combination of free-breathing navigator-gated 3D coronary magnetic resonance angiography with SENSE imaging is described. Applying a SENSE reduction factor of two at a main magnet field of 3 T allows to image long portions of the left and the right coronary artery system during half of the scan time, when compared to normal acquisition.

The present invention relates to a magnetic resonance imaging method for forming an image of an object from a plurality of signals acquired from a plurality of receiver antenna positions, wherein MR signals are measured along a predetermined trajectory containing a plurality of lines in k-space by application of a read gradient and other gradients, whereas a navigator gradient is applied for the measurement of navigator MR signals, according to the preamble portion of claim 1. The invention is further directed to a magnetic resonance apparatus and a computer program product for executing the method according to the preamble portions of claims 4 and 5, respectively.

Such a navigator-gated method is e.g. known as free-breathing navigator-gated 3D coronary magnetic resonance angiography (MRA), which has been shown to be a valuable technique for the visualization of the coronary artery system as described for example in Botnar R M et al. In Circulation 1999; 99: p. 3139-3148, in Stuber M et al. in J. Am. Coll. Cardiol. 1999; 34: p. 524-531, or in Brittain et al. MRM 1995; 33: p. 689-696. However, due to limited navigator efficiency the time to acquire one entire 3D volume is relatively long.

It is an object of the present invention to improve the above mentioned navigator-gated method by a reduction of the scan time for acquiring MR data.

This object is achieved by the magnetic resonance imaging method according to claim 1, the magnetic resonance imaging system according to claim 4 and the computer program product according to claim 5.

The present invention is based in principle on the insight, that the combination of the technique with a parallel imaging approach such as SENSE, as described by Pruessmann K P et al. in MRM 1999; 42: p. 952-962, allows for a large reduction of scan time. Applying SENSE reduces the number of phase-encoding steps and thus the inherent signal-to-noise-ratio (SNR), which will render critical in high-resolution coronary MRA. For this reason any combination of navigator-gated and corrected 3D coronary MRA with a parallel imaging like SENSE has not been envisaged feasible.

However, it was surprisingly found that compact 3Tesla whole body MR systems the intrinsic increase of SNR at higher main magnetic field of at least 2.5 Tesla is compensating the aforementioned disadvantage of the SENSE technique. It was therefore very surprisingly that coronary MRA performed on a 3Tesla system offered favorable preliminary conditions for a successful combination of coronary MRA with SENSE.

These and other aspects of the invention will be elaborated with reference to the preferred implementations as defined in the dependent claims. In the following description an exemplified embodiment of the invention is described with respect to the accompanying drawings. It shows

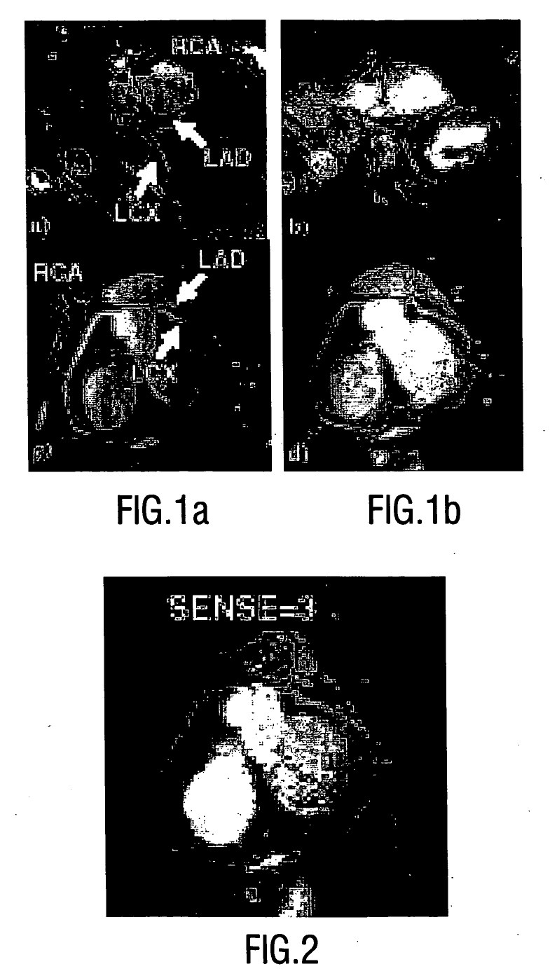

FIG. 1 the left and right coronary artery systems measured without and with SENSE acquisition,

FIG. 2 the right coronary artery acquired with a SENSE factor of 3, and

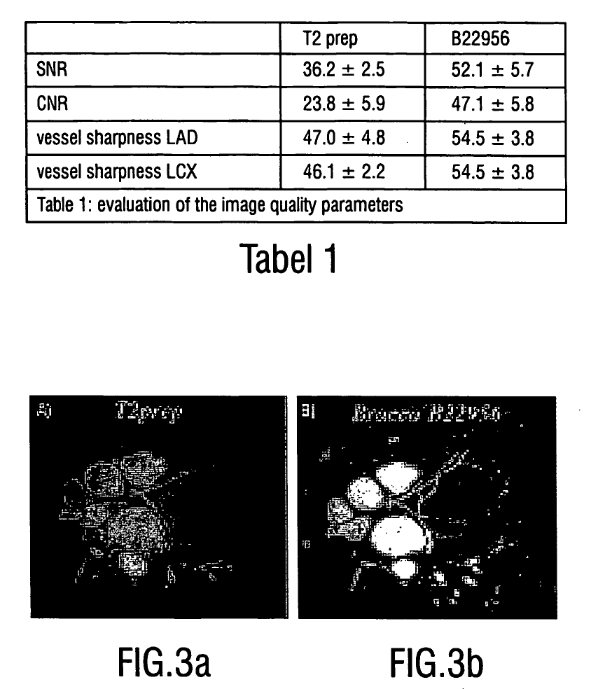

FIG. 3a multi-planar reformatted images of the T2prep acquisition and

FIG. 3b the contrast agent enhanced inversion recovery acquisition obtained in the same subject.

In the present description a multiple of receiver antenna or coils are used. However, it is also possible to implement the SENSE method with a single receiving coil or antenna at different receiving positions.

The increased signal-to-noise-ratio at a main magnetic field of 3 Tesla offers favorable conditions for parallel imaging approaches. Therefore SENSE imaging is combined with free-breathing navigator-gated and corrected 3D coronary MRA at a stationary magnetic field strength of at least 2.5 T. Both left and right coronary artery systems were successfully visualized with and without SENSE reduction in two healthy subjects. Applying SENSE enables for a scan time reduction without compromising the visibility of the coronary vessels.

Free-breathing navigator-gated and corrected double-oblique 3D coronary MRA was performed on a Philips 3T Intera whole body MR unit (Philips Medical Systems, Best, The Netherlands), equipped with a receive/transmit body coil and a vector-ECG. Two healthy adult volunteers were investigated. To allow for SENSE acquisition six coil elements were used for optimal signal reception. Two of them were positioned on the chest wall, two on the back and one on each lateral side. At the beginning of the examination a SENSE reference scan was acquired in order to determine the sensitivity of each coil. The subsequent imaging sequence parameter of the segmented k-space gradient-echo sequence included 10 excitations/R-R interval, TR=8.1 ms, TE=2.4 ms, α=30′. A field-of-view of 360×270 mm² was sampled with a 512×391 matrix. Ten slices of 3 mm thickness were acquired and interpolated (zero filling) to 20 slices. A spectrally selective fat-saturation and a T2-preparation were added in front of the acquisition part. The left and right coronary artery systems were each measured twice: with no SENSE reduction and with a reduction factor (R) of two. In one volunteer, where the navigator efficiency was above 60%, there was enough time to perform a third scan of the right coronary artery (RCA) with R=3. The SENSE fold-over direction was always chosen into antero-posterior direction. For better visualization of the vessels each 3D data set was multi-planar reformatted.

In all cases, the scans with and without SENSE could be successfully performed. In FIG. 1 multi-planer reformatted data sets acquired withhout SENSE (a, c) and aquired with SENSE with a reduction factor or two (b, d) are shown. In FIG. 2 coronary MRA acquired with a SENSE reduction factor of three is shown. All data sets were able to visualize extended portions of the left circumflex (LCX), the left anterior descending (LAD) and/or the RCA. As expected, highest subjective image quality was obtained without SENSE (FIGS. 1a,c). However, comparable image quality was also achieved with the SENSE scans for R=2 (FIGS. 1b,d), or even for R=3 (FIG. 2). Applying SENSE further enabled for a scan time reduction from approximately 12 min (no SENSE) down to 6 min (R=2), respectively 4 min (R=3), for an average navigator efficiency of 50%.

In conclusion, it is shown that 3D coronary MRA at a stationary magnetic field strength of at least 3T can successfully be combined with SENSE imaging. Applying a SENSE reduction factor of two allows to image long portions of the left and the right coronary artery system during half of the scan time, when compared to the acquisition without SENSE. Further, the image obtained with a reduction factor of three gives an indication for the potential of applying parallel imaging on a high field system.

Magnetic resonance angiography (MRA) has been shown to be a promising technique for the visualization of the proximal coronary arteries. Among others, state of the art techniques include high-resolution 3D image acquisition, navigator-based scanning during free-breathing, and preparation pulses for fat suppression and myocardial suppression (T2prep). However, a further enhanced signal and improved contrast between blood and the myocardium is desirable, thereby the application of a contrast agent can be very helpful, especially for the visualization of more distal or branching vessels. Various intravascular contrast agents are available on the market. Particular intravascular contrast agents are characterized by reduced leakage into interstitial compartments, some of them—like B-22956—also by a long plasma half life. Therefore they significantly reduce the T1 relaxation of blood and show only minor effects on the T1 relaxation of the myocardial muscle. In the present invention a 3D free-breathing navigator-gated and corrected gradient echo sequence has been adapted for contrast enhanced coronary MRA using a new intravascular, low molecular weight Gd based chelate coded B22956 1 (Bracco S.p.A., Milan, Italy). The contrast agent was applied on six healthy volunteers and objectively compared with a T2 magnetization prepared scan without contrast agent administration.

Six healthy adult subjects were studied on a commercial 1.5T Philips Gyroscan ACS-NT system (Philips, Best, The Netherlands), equipped with a 5-element cardiac synergy-coil and a vector ECG. For each volunteer, double oblique 3D coronary MRA was performed 5 minutes after the contrast agent was administered to the human body. For comparison reason baseline coronary MRA with T2 preparation 2.3 (T2prep) were performed prior to the contrast agent scan. The imaging sequence post contrast was a free-breathing navigator-gated 3D segmented k-space gradient echo sequence. A field-of-view of 360 mm was sampled with a 512×512 matrix resulting in an in-plane spatial resolution of 0.7×0.7 mm². Ten 3 mm slices were acquired and interpolated to twenty with a thickness of 1.5 mm. TR was 7.5 ms and TE 2.1 ms. For non-contrast enhanced baseline coronary MRA, a T2prep was used and an inversion recovery pre-pulse was added to the contrast agent scan. The time delay between the inversion and the image acquisition part was adjusted in order to null the myocardial signal (TI=180 ms). Since the inversion pre-pulse is non-slice selective, it also affects the magnetization of the diaphragm used as an interface for navigator gating. Therefore a navigator restoring pulse (NavRestore—see Stuber et. al., MRM 45:p. 206-211 (2001)) was implemented, which locally re-inverts the longitudinal magnetization of the navigator kernel immediately after the inversion pre-pulse. Prior to each navigator-gated scan, a preparation phase of 25 RF excitations is performed for the correct determination of the most cranial end-expiratory position of the diaphragm (=navigator preparation phase). In the subsequent acquisition part, this end-expiratory position is used to calculate the relative respiratory diaphragmatic displacement. In order to have similar magnetization conditions on the diaphragm during the preparation phase and during the actual imaging phase, the pre-pulses (T2Prep for non-contrast agent scan; inversion recovery and NavRestore for contrast agent scan) were also performed during the ECG triggered navigator preparation phase. For the contrast-enhanced examination 0.075 mmol/kg-body weight of B22956 was administered intravenously over 150 s. Data acquisition was started 5 min after the end of the contrast agent injection. The evaluation of SNR, CNR and vessel sharpness was subsequently performed for the objective comparison of the pre- and post-contrast scans.

The application of an inversion recovery pre-pulse very efficiently suppressed the myocardial muscle. While the increased relaxivity of the blood allowed for a good visualization of the coronaries, even for more distal segments or branching vessels In FIG. 3a multi-planar reformatted images of the T2prep acquisition are shown and in FIG. 3b the contrast agent enhanced inversion recovery acquisition obtained in the same subject. A comparison of the objectively determined image quality parameters shows increased values for the contrast-enhanced coronary MRA (Table 1). The acquisition with B22956 in conjunction with the inversion recovery sequence resulted in a significant increase of SNR (39%, P<0.05), CNR (95%, P<0.01) and vessel sharpness (19%, P<0.05) when compared with the T2prep non-contrast enhanced scan. Due to its high proportion of biliary excretion, administration of B-22956 together with the NavRestore resulted in a high liver signal which facilitated the use of real-time navigator technology. For the determination of the end-expiratory position, the preparation phase of the navigator is performed triggered an with the application of the magnetization pre-pulses: in FIG. 3a with T2prep sequence and in FIG. 3b with B22956 with inversion recovery and NavRestore pre-pulses. Further, the application of the magnetization preparation pulses (T2prep resp. Inversion and NavRestore) during the triggered navigator preparation phases ensured a reliable determination of the end-expiratory diaphragmatic position, during both preparation and scanning phase.

It can be concluded that the new contrast agent B22956 has been successfully combined with a free-breathing navigator-gated and corrected inversion recovery 3D coronary MRA. The reduced T1 of the blood and the good intravascularity of the agent enables the visualization of more distal segments and branching vessels of the left coronary arterial system while myocardial muscle signal was almost entirely suppressed.

The magnetic resonance imaging system includes a set of main coils whereby a steady, uniform magnetic field is generated. The main coils are constructed, for example in such a manner that they enclose a tunnel-shaped examination space. The patient to be examined is slid on a table into this tunnel-shaped examination space. The magnetic resonance imaging system also includes a number of gradient coils whereby magnetic fields exhibiting spatial variations, notably in the form of temporary gradients in individual directions, are generated so as to be superposed on the uniform magnetic field. The gradient coils are connected to a controllable power supply unit. The gradient coils are energized by application of an electric current by means of the power supply unit. The strength, direction and duration of the gradients are controlled by control of the power supply unit. The magnetic resonance imaging system also includes transmission and receiving coils for generating RF excitation pulses and for picking up the magnetic resonance signals, respectively. The transmission coil is preferably constructed as a body coil whereby (a part of) the object to be examined can be enclosed. The body coil is usually arranged in the magnetic resonance imaging system in such a manner that the patient to be examined, being arranged in the magnetic resonance imaging system, is enclosed by the body coil. The body coil acts as a transmission aerial for the transmission of the RF excitation pulses and RF refocusing pulses. Preferably, the body coil involves a spatially uniform intensity distribution of the transmitted RF pulses. The receiving coils are preferably surface coils which are arranged on or near the body of the patient to be examined. Such surface coils have a high sensitivity for the reception of magnetic resonance signals which is also spatially inhomogeneous. This means that individual surface coils are mainly sensitive for magnetic resonance signals originating from separate directions, i.e. from separate parts in space of the body of the patient to be examined. The coil sensitivity profile represents the spatial sensitivity of the set of surface coils. The transmission coils, notably surface coils, are connected to a demodulator and the received magnetic resonance signals (MS) are demodulated by means of the demodulator. The demodulated magnetic resonance signals (DMS) are applied to a reconstruction unit. The reconstruction unit reconstructs the magnetic resonance image from the demodulated magnetic resonance signals (DMS) and on the basis of the coil sensitivity profile of the set of surface coils. The coil sensitivity profile has been measured in advance and is stored, for example electronically, in a memory unit which is included in the reconstruction unit. The reconstruction unit derives one or more image signals from the demodulated magnetic resonance signals (DMS), which image signals represent one or more, possibly successive magnetic resonance images. This means that the signal levels of the image signal of such a magnetic resonance image represent the brightness values of the relevant magnetic resonance image. The reconstruction unit in practice is preferably constructed as a digital image processing unit which is programmed so as to reconstruct the magnetic resonance image from the demodulated magnetic resonance signals and on the basis of the coil sensitivity profile. The digital image processing unit is notably programmed so as to execute the reconstruction in conformity with the so-called SENSE technique or the so-called SMASH technique. The image signal from the reconstruction unit is applied to a monitor so that the monitor can display the image information of the magnetic resonance image (images). It is also possible to store the image signal in a buffer unit while awaiting further processing, for example printing in the form of a hard copy.

In order to form a magnetic resonance image or a series of successive magnetic resonance images of the patient to be examined, the body of the patient is exposed to the magnetic field prevailing in the examination space. The steady, uniform magnetic field, i.e. the main field, orients a small excess number of the spins in the body of the patient to be examined in the direction of the main field. This generates a (small) net macroscopic magnetization in the body. These spins are, for example nuclear spins such as of the hydrogen nuclei (protons), but electron spins may also be concerned. The magnetization is locally influenced by application of the gradient fields. For example, the gradient coils apply a selection gradient in order to select a more or less thin slice of the body. Subsequently, the transmission coils apply the RF excitation pulse to the examination space in which the part to be imaged of the patient to be examined is situated. The RF excitation pulse excites the spins in the selected slice, i.e. the net magnetization then performs a precessional motion about the direction of the main field. During this operation those spins are excited which have a Larmor frequency within the frequency band of the RF excitation pulse in the main field. However, it is also very well possible to excite the spins in a part of the body which is much larger man such a thin slice; for example, the spins can be excited in a three-dimensional part which extends substantially in three directions in the body. After the RF excitation, the spins slowly return to their initial state and the macroscopic magnetization returns to its (thermal) state of equilibrium. The relaxing spins then emit magnetic resonance signals. Because of the application of a read-out gradient and a phase encoding gradient, the magnetic resonance signals have a plurality of frequency components which encode the spatial positions in, for example the selected slice. The k-space is scanned by the magnetic resonance signals by application of the read-out gradients and the phase encoding gradients. According to the invention, the application of notably the phase encoding gradients results in the sub-sampling of the k-space, relative to a predetermined spatial resolution of the magnetic resonance image. For example, a number of lines which is too small for the predetermined resolution of the magnetic resonance image, for example only half the number of lines, is scanned in the k-space.

1. Magnetic resonance imaging method for forming an image of an object from a plurality of signals acquired from a plurality of receiver antenna positions, wherein MR signals are measured along a predetermined trajectory containing a plurality of lines in k-space by application of a read gradient and other gradients, whereas a navigator gradient is applied for the measurement of navigator MR signals, wherein phase corrections are determined from phases and moduli of the navigator MR signals so as to correct the measured MR signals, and an image of a part of the object is determined from the corrected MR signals, each receiver antenna position having a spatial sensitivity profile, and a magnetic resonance image is reconstructed from the undersampled magnetic resonance signals, the spatial sensitivity profiles of each receiver antenna position and the navigator signals:

2. Magnetic resonance imaging method according to claim 1, whereas the method is performed at a high stationary magnetic field strength of at least 2.5-T.

3. Magnetic resonance imaging method according to claim 1, whereas the method is performed in 3D coronary magnetic resonance angiography.

4. A magnetic resonance imaging system comprising.
a static main magnet having a main magnetic field,
at least one receiver antenna having a plurality of receiver antenna positions,
means for applying a read gradient and other gradients,
means for measuring MR signals along a predetermined trajectory containing a plurality of lines in k-space
means for applying a navigator gradient for the measurement of navigator MR signals,
means for determining phase corrections from phases and moduli of the navigator MR signals so as to correct the measured MR signals
means for determining an image of a part of the object from the corrected MR signals
a receiver antenna system for acquiring undersampled MR signals, each receiver antenna position having a spatial sensitivity profile,
means for reconstruction of magnetic resonance images from the undersampled MR signals, the spatial sensitivity profiles of each receiver antenna position and the navigator signals.

5. A computer program product stored on a computer usable medium for forming an image by means of a magnetic resonance method comprising a computer readable program means for causing the computer to control the execution of:
applying a read and other gradients,
measuring MR signals along a predetermined trajectory containing a plurality of lines in k-space
applying a navigator gradient for the measurement of navigator MR signals,
determining phase corrections from phases and moduli of the navigator MR signals so as to correct the measured MR signals
determining an image of a part of the object from the corrected MR signals
acquiring undersampled MR signals from a receiver antenna system, each receiver antenna position having a spatial sensitivity profile,
reconstruction of magnetic resonance images from the undersampled MR signals, the spatial sensitivity profiles of each receiver antenna position and the navigator signals.

6. Magnetic resonance imaging system method as claimed in claim 1 wherein an intravascular contrast agent with a reduced leakage into interstitial compartments is administered in the human body before imaging.

7. Magnetic resonance imaging method according to claim 6, wherein the contrast agent has a long plasma half life.

8. Magnetic resonance imaging method according to claim 6, wherein the contrast agent is low molecular weight Gd based chelate.
